(19) United States
(12) Patent Application Publication
(10) Pub. No.: US 2024/0065531 A1
(43) Pub. Date: Feb. 29, 2024

(54) HAND-HELD ENDOSCOPIC SYSTEM WITH A SINGLE-USE CANNULA AND A SINGLE-USE ACCESS SHEATH

(71) Applicant: MicronVision Corp., Bellevue, WA (US)

(72) Inventor: Xiaolong OUYANG, Bellevue, WA (US)

(21) Appl. No.: 18/374,740

(22) Filed: Sep. 29, 2023

Related U.S. Application Data
(63) Continuation-in-part of application No. 18/083,209, filed on Dec. 16, 2022.
Application No. 18/083,209 is a continuation-in-part of application No. 17/835,624, filed on Jun. 8, 2022, now Pat. No. 11,684,248.
(60) Provisional application No. 63/411,157, filed on Sep. 29, 2022.
(60) Provisional application No. 63/432,980, filed on Dec. 15, 2022.
(60) Provisional application No. 63/434,176, filed on Dec. 21, 2022.
(60) Provisional application No. 63/437,462, filed on Jan. 6, 2023.
(60) Provisional application No. 63/440,151, filed on Jan. 20, 2023.
(60) Provisional application No. 63/442,800, filed on Feb. 2, 2023.
(60) Provisional application No. 63/446,416, filed on Feb. 17, 2023.
(60) Provisional application No. 63/446,807, filed on Feb. 18, 2023.
(60) Provisional application No. 63/449,168, filed on Mar. 1, 2023.
(60) Provisional application No. 63/451,595, filed on Mar. 12, 2023.
(60) Provisional application No. 63/454,640, filed on Mar. 25, 2023.
(60) Provisional application No. 63/454,953, filed on Mar. 27, 2023.
(60) Provisional application No. 63/460,728, filed on Apr. 20, 2023.
(60) Provisional application No. 63/461,939, filed on Apr. 26, 2023.
(60) Provisional application No. 63/462,985, filed on Apr. 29, 2023.
(60) Provisional application No. 63/466,318, filed on May 14, 2023.
(60) Provisional application No. 63/521,704, filed on Jun. 19, 2023.
(60) Provisional application No. 63/522,395, filed on Jun. 21, 2023.
(60) Provisional application No. 63/524,951, filed on Jul. 5, 2023.
(60) Provisional application No. 63/535,077, filed on Aug. 29, 2023.
(60) Provisional application No. 63/536,700, filed on Sep. 5, 2023.
(60) Provisional application No. 63/417,340, filed on Oct. 19, 2022.

(51) Int. Cl. A61B 1/00; A61B 1/015; A61B 1/05; A61B 1/307; A61M 25/00

(52) U.S. Cl. CPC A61B 1/00103; A61B 1/000095; A61B 1/000096; A61B 1/00087; A61B 1/00135; A61B 1/015; A61B 1/05; A61B 1/307; A61M 25/0084

(57) ABSTRACT

A portable endoscopic system injects medication into a patient's cavity using a single-use access sheath inserted in the cavity with a single-use endoscopic cannula inserted in the sheath. A display mounted on a handle secured to the cannula shows images of target tissue taken with an imaging module at a distal end of the cannula. The sheath can remain in the same position in the patient while the cannula rotates in the sheath. The distal end of the injection needle is offset radially from a central axis of the cannula, so rotation of the cannula points the needle to new injections sites. The cannula, needle, and injection sites are seen on the display, as is the angular position of the cannula relative to markings at the distal end of the sheath. The distal end of the sheath has surfaces made of or covered by non-reflective or low-reflection material to prevent or reduce image artifacts due to reflection from the sheath.

### REFERENCE TO RELATED APPLICATIONS

This application claims the benefit of and incorporate by reference each of the following U.S. provisional patent applications:
Ser. No. 63/411,157 filed Sep. 29, 2022
Ser. No. 63/432,980, filed Dec. 15, 2022
Ser. No. 63/434,176, filed Dec. 21, 2022
Ser. No. 63/437,462, filed Jan. 6, 2023
Ser No. 63/440,151, filed Jan. 20, 2023
Ser. No. 63/442,800, filed Feb. 2, 2023
Ser. No. 63/446,416, filed Feb. 17, 2023
Ser. No. 63/446,807, filed Feb. 18, 2023
Ser. No. 63/449,168, filed Mar. 1, 2023
Ser. No. 63/451,595, filed Mar. 12, 2023
Ser. No. 63/454,640, filed Mar. 25, 2023
Ser. No. 63/454,953, filed Mar. 27, 2023
Ser. No. 63/460,728, filed Apr. 20, 2023
Ser. No. 63/461,939, filed Apr. 26, 2023
Ser. No. 63/462,647, filed Apr. 28, 2023
Ser. No. 63/462,985, filed Apr. 29, 2023
Ser. No. 63/466,318, filed May 14, 2023
Ser. No. 63/521,704, filed Jun. 19, 2023
Ser. No. 63/522,395, filed Jun. 21, 2023
Ser. No. 63/524,951, filed Jul. 5, 2023
Ser. No. 63/535,077 filed Aug. 12, 2023
Ser. No. 63/535,077, filed Aug. 29, 2023
Ser. No. 63/536,700, filed Sep. 5, 2023

This patent specification is a continuation in-part of each of U.S. patent application Ser. No. 18/083,209 filed Dec. 16, 2022 and Ser. No. 17/835,624 filed Jun. 8, 2022, claims the benefit of the filing date of each and incorporates by reference the subject matter of each.

### FIELD

This patent specification relates to portable, hand-held endoscopes that are entirely or partly single-use.

### BACKGROUND

Portable, hand-held endoscopes with single-use portions that contact the patient find wider use in medical procedures and gradually supersede conventional endoscopes that are much more expensive and require thorough decontamination between patients that entails use of specialist personnel and facilities for decontamination.

Hand-held endoscopes are referred to in the following U.S. Patents and U.S. Published Patent Applications, which are hereby incorporated by reference: US-20230200634-A1, US-20230128303-A1, US-20230117151-A1, US-20220296090-A1, US-20220273165-A1, US-20220211252-A1, US-20220142460-A1, US-20220079418-A1, US-20210401277-A1, US-20210338052-A1, US-20210307591-A 1, US-20210251789-A1, US-20200221932-A1, US-20200077880-A1, US-20190282071-A1, US-20190216325-A1, US-20180256009-A1, US-20180132700-A1, US 20170215699-A1, US-20170188793-A1, US-20170188795-A1, US-20160367119-A1, US-20160174819-A1, US-20150164313-A1, US-20150157387-A1, US-20150150441-A1, US-20120289858-A1, US-20120100729-A1, US-20110270179-A1, US-20110009694-A1, US-20100284580-A1, US-20100286477-A1, US-20100121139-A1, US-20100121142-A1, US-20100121155-A1, US-20110009694-A1, US20110006364-A1, U.S. Pat. No. 11,684,248-B2, U.S. Pat. No. 11,395,579-B2, US-20220168035-A1 U.S. Pat. No. 11,330,973-B2, U.S. Pat. No. 11,071,442-B2, U.S. Pat. No. 11,013,396-B2, US-20210137352-A1, US-20210093169-A1, U.S. Pat. No. 10,874,287-B2, U.S. Pat. No. 10,869,592-B2, U.S. Pat. No. 10,524,636-B2, U.S. Pat. No. 10,441,134-B2 U.S. Pat. No. 10,426,320-B2, US-20190261836-A1, U.S. Pat. No. 10,362,926-B2, U.S. Pat. No. 10,292,571-B2, U.S. Pat. No. 10,278,563-B2, US-20190117050-A1, US-20180271581-A1 US-20180256009-A1, U.S. Pat. No. 10,045,686-B2, US-20180206707-A9, US-20180132701-A1 U.S. Pat. No. 9,895,048-B2, US-20170188794-A1, US-20170188795-A1, U.S. Pat. No. 9,649,014-B2. U.S. Pat. No. 9,622,646-B2, U.S. Pat. No. 9,468,367-B2, US-20140288460-A1, US-20140276207-A1, US-20130296648-A1, U.S. Pat. No. 8,460,182-B2

### SUMMARY

According to some embodiments, a portable endoscopic system with a single-use cannula and a single-use access sheath comprises: a single-use, tubular access sheath that has open proximal and distal ends; a single-use cannula that has a distal portion releasably sliding axially in said sheath to a working position in the sheath in which a distal end of the cannula is at a distal end of but inside the sheath and a proximal portion of the cannula extends proximally from the sheath; wherein the outer diameter of the cannula portion that is in the sheath is sufficiently smaller than the inner diameter of the surrounding sheath to leave space 803 for axial fluid flow; wherein said sheath has: sheath ports and at its proximal and distal portions, respectively; a back-flow restrictor at its proximal end configured to resist proximal fluid flow from said space between the cannula and the sheath; physical markings at its distal end arranged at selected angularly spaced positions; and a funnel-shaped entrance into its open proximal end; wherein said cannula has: an imaging module at its distal end configured to produce image data for a field of view that includes said markings and further includes an imaged region distal from the sheath; a permanently built-in, hollow injection needle inside the cannula that has a distal end configured to move between an extended position in which it extend distally from the sheath and a retracted position in which it does not; cannula ports at proximal and distal portions of the cannula, respectively, and one or more internal channels coupling the cannula ports for fluid flow; a syringe port at the proximal portion of the cannula, coupled for fluid flow to a proximal end of the injection needle; a syringe body coupled with the injection needle and configured to move the needle axially between said retracted and extended positions thereof; and a needle position lock configured to selectively lock the injection needle against axial motion.

According to some embodiments, the portable endoscopic system includes one of more of the following: (a) the open distal end of the sheath has a radially inwardly facing surface that is made of or is covered with a material characterized by low light reflectivity to thereby prevent or at least significantly reduce artifacts in images from said imaging module due to light reflected by said radially inwardly facing surface of the sheath; (b) the open distal end of the sheath has distally facing surface 104E that is made of or covered with a material characterized by low light reflectivity to thereby prevent or at least significantly reduce artifacts in images from said imaging module due to light reflected by said distally facing distal face of the sheath; (c) an aspiration source and an aspiration tube operatively coupling the aspiration source and said sheath port at said proximal portion of the sheath, and a flow regulator operatively coupled with said aspiration tube to selectively close said aspiration tube partly or completely; (d) said flow regulator can comprise a hand operated wheel 336 and an inclined raceway along which the wheel moves axially to thereby press the aspiration tube to selected degrees and restrict flow therethrough to a selected degree; (e) said flow regulator can comprise a hand operated stopcock; (f) said sheath further includes a tubular cap body at said open proximal end of the sheath and a lid hinged to the cap body to selectively close the cap body or open the cap body for passage therethrough of said cannula; (g) a reusable portion to which said cannula mounts releasably and which comprises a display operatively coupled with said imaging module to display images taken with the imaging module, and a handle on which the display is mounted; (h) a distal end of said injection needle is offset from a central axis of said cannula and said cannula is configured to rotate in said access sheath which the sheath remains stationary relative to space of to a patient, wherein the needle points to different injection sites for different angular positions of the cannula relative to the sheath; (i) the portion of the access sheath between the funnel-shaped proximal end and the distal end thereof is approximately 10 cm; and (j) the cannula portion that is in the access sheath is no longer than 11.5 cm.

According to some embodiments, a portable endoscopic system with a single-use cannula and a single-use access sheath comprises: a single-use, tubular access sheath that has open proximal and distal ends; a single-use cannula that has a distal portion releasably sliding axially in said sheath to a working position in which a distal end of the cannula is at a distal end of but inside the sheath and a proximal portion of the cannula extends proximally from the sheath; wherein the outer diameter of the cannula portion that is in the sheath is sufficiently smaller than the inner diameter of the surrounding sheath to leave space 803 for axial fluid flow; wherein said sheath has: sheath ports and at its proximal and distal portions, respectively; and physical markings at its distal end arranged at selected angularly spaced positions; wherein said cannula has: an imaging module at its distal end configured to produce image data for a field of view that includes said markings and further includes a target region distal from the sheath; a hollow injection needle inside the cannula that has a distal end configured to move between an extended position in which it extend distally from the sheath and a retracted position; cannula ports at proximal and distal portions of the cannula, respectively, and one or more internal channels coupling the cannula ports for fluid flow; a syringe port at the proximal portion of the cannula, coupled for fluid flow to a proximal end of the injection needle; and a needle position lock configured to selectively lock the injection needle against axial motion.

According to some embodiments, the portable endoscopic system described in the immediately preceding paragraph includes one of more of the following: (a) the open distal end of the sheath has a radially inwardly facing surface that is made of or is covered with a material characterized by low light reflectivity to thereby prevent or at least significantly reduce artifacts in images from said imaging module due to light reflected by said radially inwardly facing surface of the sheath; (b) the open distal end of the sheath has a distally facing surface that is made of or covered with a material characterized by low light reflectivity to thereby prevent or at least significantly reduce artifacts in images from said imaging module due to light reflected by said distally facing surface of the sheath; (c) a syringe body connects to a proximal end of said injection needle and is configured for manual operation to move said needle axially between said extended and retracted position; (d) said cannula is configured to rotate relative to the access sheath; and (e) the distal end of said injection needle is offset from a central axis of the cannula and the cannula is configured for rotation in the axial sheath, whereby different angular position of the cannula relative to the sheath cause the distal end of the needle to point at different injection sites.

According to some embodiments, a method of injecting medication in a patient's cavity under endoscopic guidance comprises: introducing an access sheath into the patient's cavity; observing tissue distal of a distal end of the access sheath on a display mounted on a handle secured to a proximal end of a cannula, wherein an imaging module at a distal end of the cannula inserted in the access sheath provides image data to said display; selectively flushing the cavity with a fluid entering the cannula through a port at a proximal portion thereof and exiting the cannula at one or more ports at a distal portion thereof with flushing fluid passing through one or more internal channels in the cannula; selectively aspirating the cavity through ports at a distal portion of the access sheath, a space between the cannula and the access sheath, and a port at a proximal portion of the access sheath; injecting medication at a first target site in the cavity with an injection needle that is permanently build inside the cannula, moves axially between extended and retracted positions, and has a distal end offset radially from a central axis of the cannula; and rotating the cannula relative to the sheath to thereby point a distal end of the injection needle at a second injection site and injecting medication at the second injection site.

According to some embodiments, the method can further include one or more of the following: (a) the introducing step comprises introducing an access sheath with open distal end that has a radially inwardly facing surface made of or covered with a material characterized by low light reflectivity to thereby prevent or at least significantly reduce artifacts in images from said imaging module due to light reflected by said radially inwardly facing surface of the sheath; (b) the introducing step comprises introducing an access sheath with an open distal end that has a distally facing surface made of or covered with a material characterized by low light reflectivity to thereby prevent or at least significantly reduce artifacts in images from said imaging module due to light reflected by said distally facing surface of the sheath; (c) observing angular position of the cannula relative to the sheath on the display indicated by images of markings angularly spaced around a distal end of the sheath that is within the field of view of the imaging module; (d) the sheath remains at the same angular position relative to the cavity while the cannula rotates between the first and second injections; (e) the sheath remains with said port at the proximal portion of the sheath pointing down during the first and the second injections; and (f) the sheath is inserted in a patient's urethra to the patient's bladder.

The Summary pertains to the subject matter of the initially presented claims, which may change during prosecution of this patent application.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through the accompanying drawings in which:

FIG. 1 is a perspective view illustrating a portable, hand-held endoscopic system in use for injecting medication in a target region in a patient's cavity, according to some embodiments.

FIG. 2 is a perspective view of an endoscopic system showing a flow regulator, according to some embodiments.

FIG. 2A is a perspective view of an endoscopic system showing a different flow regulator, according to some embodiments.

FIG. 3 is a perspective view of a single-use cannula and a single-use access sheath, according to some embodiments.

FIG. 4 is a perspective view of an access sheath and a flow controller and of a detail of a distal end of the sheath, according to some embodiments.

FIG. 4A is a perspective view of a sheath illustrating another example of a flow regulator that controls flow through a tube to an aspiration port in a sheath, and also illustrates distal surfaces of the sheath that are made of or are covered with a material characterized by low reflectivity of light, according to some embodiments.

FIG. 4B schematically illustrates an alternative or additional way to eliminate or at least substantially reduce effects of reflection from a radially inwardly facing surface of an access sheath, according to some embodiments.

FIGS. 5A-5E illustrate a cap closing a proximal end of an access sheath before a medical procedure, with a lid that opens for insertion of a single-use cannula, according to some embodiments.

FIG. 6 is a perspective view of a distal portion of a cannula in an access sheath, according to some embodiments.

FIGS. 7A-7H show several views of an access sheath, according to some embodiments.

FIG. 7I shows a sectional view of an access sheath with a duckbill valve for preventing backflow.

FIGS. 8A-8D show several views of an access sheath and a cannula, with examples of dimensions, and show in cross-section a space for fluid flow between a cannula and an access sheath, according to some embodiments.

FIGS. 9A-9C show a cannula in an access sheath, a flow controller, and examples of dimensions, according to some embodiments.

FIGS. 10A and 10B show an access sheath with other examples of dimensions, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein.

Individual features, components, and elements of one or several of the specific embodiments described herein can be used in or in combination with other described embodiments or with other features, components, and elements. Like reference numbers and designations in the various drawings indicate like elements.

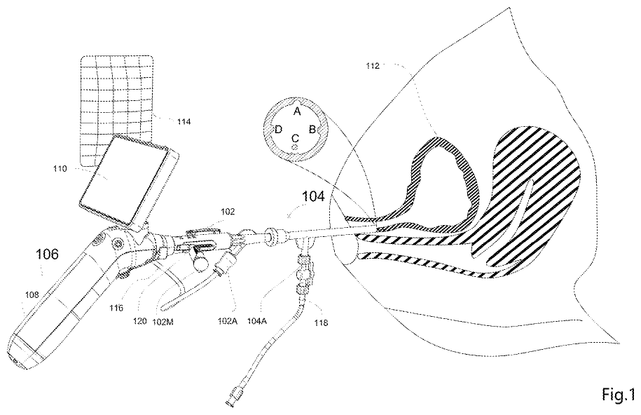

FIG. 1 illustrates a portable, hand-held endoscopic system 100 that includes a single-use cannula 102, a single-use access shield 104 into which a distal portion of cannula 102 is inserted, and a reusable portion 106 to which cannula 102 is releasably mounted, according to some embodiments. Reusable portion 106 comprises a handle 108 and a display 110. Access sheath 104 has an outflow port 104A near its proximal end. Cannula 102 has an inflow port 102A proximal to access sheath 104 and a syringe port 102M near its proximal end. Cannula 102 and access sheath 104 have ports near their distal ends that are shown in other figures. In addition, cannula 102 encloses an internal, permanently built-in retractable injection needle, an internal lumen for fluid flow, and space is left between the cannula and the access sheath for fluid flow along the length of the cannula portion that is inside the access sheath.

In a typical medical procedure, a user removes the cannula and access sheath from a sterile package, fully inserts cannula 102 in sheath 104, and inserts sheath 104 in a patient passageway such as the urethra to a desired position relative to the bladder 112. Typically, before this insertion the user connects cannula 102 to reusable portion 106 so that the insertion is guided by images taken with an imaging module at the distal end of cannula 102 observed on display 102. Before or after the insertion, the user connects a fluid source 114 to inflow port 102A through a tube 116, connects an aspiration source (not shows in FIG. 1) to outflow port 104A through a tube 118, and connects a medication source (not shown in FIG. 1) such a s syringe to medication port 102M.

The user selects an injection site while observing display 114, advances the injection needle distally out of the distal end of sheath 104 by pushing syringe body 120, and injects medication into the patient tissue. Syringe body 120 can be a tube configured to accept a conventional syringe for injecting medication into port 102M and thus into injection needle 126, or as a specialized syringe into which medication can be inserted and injected into needle 126. Typically, the user then rotates cannula 102 in access sheath 104 while the access sheath remains in the same position in the patient to select another injection site. The injection needle is offset from the central axis of the sheath so rotating the cannula points the needle to a different point. The user may repeat this several times to inject medication in several locations. If desired, the user may move the entire endoscopic system distally to inject medication into different tissue.

Shown as a blow-up detail in FIG. 1 is a view in the proximal direction of the distal end of access sheath 104. Physical markings ABCD are within the field of view of the imaging module, as is the portion of the injection needle protruding from access sheath 104. These markings and the injection needle are seen on display 100 to help guide the selection of injection sites as the user rotates cannula 102 in access sheath 104 as they allow visualization of the cannula orientation while the user is focused on the images on display 110.

Notably, access sheath 104 need not be moved when the user repositions the injection needle for injecting at different sites. This is unlike known prior injection systems in which a cannula is in direct contact with patient tissue when rotated or an access sheath is rotated and can cause patient discomfort. The markings ABCD are particularly beneficial in accurately selecting injection sites. Preferably, access sheath 104 is orient3edwith outflow port facing down so marking A is on top, marking B is on the bottom, and markings B and D are horizontally spaced as seen on display 110.

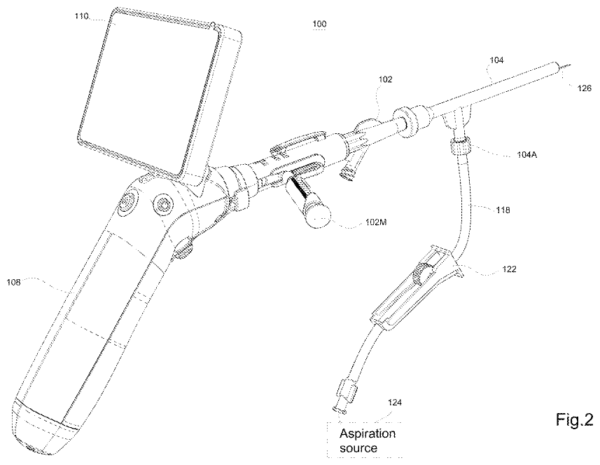

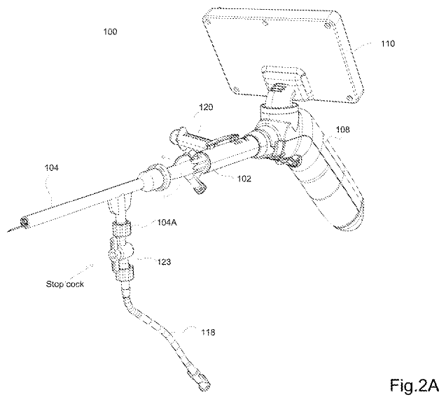

FIG. 2 is a perspective view of endoscopic system 100 showing a flow regulator 122 configured to control fluid flow through tube 118, which is connected to an aspiration source 124. FIG. 2 shows a distal end of an injection needle 126 protruding distally from the distal end of sheath 104, according to some embodiments. Handle 108 and display 110 can be like reusable portion 102 shown in FIG. 1 of U.S. Pat. No. 10,278,563, which is hereby incorporated by reference in this patent specification.

FIG. 2A is otherwise like FIG. 2 but shows a different flow regulator 123 in the form of a hand-operated stop cock 123 through which tube 118 passes. Stopcock 123 is configured to squeeze tube 118 as desired to regulate flow therethrough from full, unobstructed flow to no flow and intermediate degrees of flow. FIG. 2A also illustrates that cannula 102, which includes syringe body 120 rotates relative to handle 108 and access sheath 104, and that typically tube 118 hangs down from access sheath 104 and the access sheath remains in the illustrates angular position during a medical procedure.

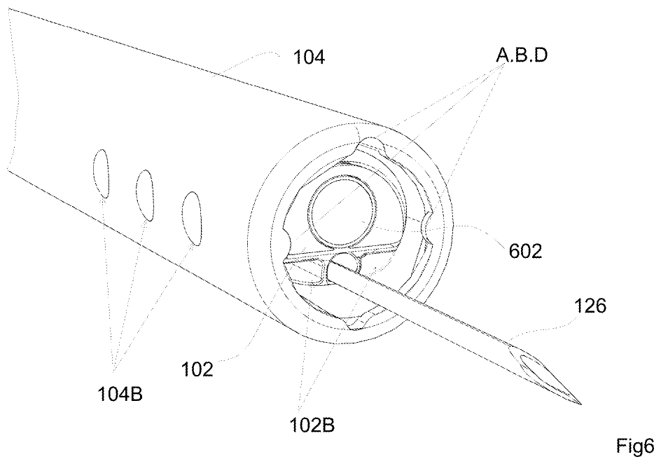

FIG. 3 is a perspective view of cannula 102 in sheath 104 and shows flow regulator 122 in more detail. The portion of cannula 102 that is proximal from access sheath 104 can be like the single-use portion 104 shown in FIG. 1 of said U.S. Pat. No. 10,278,563 and can be releasably mounted to handle 108 like said single-use portion 104 mounts to reusable portion 102 in FIG. 1 of said U.S. Pat. No. 10,278,563. Electrical connector 328 mates with an electrical receptacle in handle 108 and a mechanical connector 330 slides into and releasable locks into a mechanical receptor in handle 108 The connection establishes communication between an imaging module 602 (FIG. 6) at the distal end of cannula 102 and display 108. The portion of cannula 102 that is inside access sheath 104 is described in more detail in connection with FIG. 6, and the imaging module operates like module in subassembly 110 described in said U.S. Pat. No. 10,278,563. Duct 120 connects to a proximal end of built-in injection needle 126 and connects syringe port 102M to hollow injection needle 126. Moving duct 120 axially moves needle 126 axially between a retracted position in which the needle is inside sheath 104 and an extended position in which the needle protrudes a few mm distally from sheath 104. A needle position lock 332 is biased to interact with a holder affixed to the proximal end of needle 126 to releasably lock needle 1026 in its retracted and extended positions as well as in one or more intermediate positions, as described in more detail in said U.S. Pat. No. 10,278,563 for a like configuration.

Flow regulator 122 comprises a cradle 334 in which tube 118 is held under a wheel 336. Wheel 336 rides axially in an inclined raceway 338 between a closing position in which it compresses tube 118 to prevent flow and an open position in which it allows high flow through tube 118. In intermediate positions, wheel 336 allows intermediate amounts of flow. The user turns wheel 336 manually.

FIG. 4 is a perspective view of access sheath 104, tube 118, and flow regulator 122 and shows a blow-up detail of the distal end of access sheath 104, according to some embodiments. Sheath 104 is open at both axial ends, and its proximal end is funnel-shaped, facilitating insertion of cannula 102 therein. The physical markings ABCD are labeled. The distal periphery of access sheath is rounded to reduce patient discomfort when sheath 104 is being introduced in a passageway such as the urethra. The distal fact of access sheath 104 curves inwardly to keep the distal end of cannula 102 just proximal from the protrusions forming marking C and D.

FIG. 4A is a perspective view of sheath 104 and tube 118 and shows another example of a flow regulator—a stopcock 123. As illustrated, tube 118 connects outflow port 104A to an aspiration source 124 (FIG. 2), and flow regulator 123 in this example is a stopcock that straddles tube 118. A user controls flow through tube 118 by rotating a wing 123A to thereby squeeze tube 118 between allowing full flow, no flow, and intermediate amounts of flow.

FIG. 4A further illustrates in a blow-up that the distal end of sheath 104 has two surfaces that are distal from the imaging module 602 (FIG. 6) of a cannula 102 inserted in sheath 104. They are: (1) a radially inwardly facing surface 104D and (2) a distally facing surface 104E. At least surface 104D is made of or is covered with a material that is characterized by low reflectivity of light. Preferably, surface 104E also is made of or covered with such material. Examples of suitable material are black non-shiny plastic and flat black paint.

It has been unexpectedly discovered that reflection of light from imaging module 602 by one or both of surfaces 104D and 104E can create undesirable artifacts in images taken with imaging module 602 and displayed on display 114. Light emitted from a light source such as an LED in imaging module 602 can reflect from surface 104D and cause an image artifact in the form of a ring that is brighter than it should be. Such light reflected from surface 104D can undesirably affect the operation of Automatic Exposure Control (AEC), Automatic Gain Control (AGC), or Automatic Light Control (ALC) functions of imaging module 602. Eliminating or at least significantly reducing such light reflection from surface 104D can prevent or at least significantly reduce such artifacts and thereby improve the diagnostic value or images on display 114.

FIG. 4B schematically illustrates an alternative or additional way to eliminate or at least substantially reduce effects of such reflection from radially inwardly facing surface 104D. It has been discovered that the artifact due to such reflection is a brighter ring in the image that imaging module 602 observes, and the ring shape corresponds to the ring shape of surface 104D. As illustrated in FIG. 4B, an artificial intelligence (AI) processor receives image data from imaging module 602, carries out AI processing of the received image data to identify the ring artifact by its shape. The size of the ring artifact may vary somewhat, depending on the distance from the imaging module to the tissue being imaged but the shape remains a ring shape. AI image processor 4102 can use known technology to identify the brighter ring in the image from imaging module 602. For example, image processor 602 can store a great number of pairs of images, one with and one without a ring artifact. AI image processor 402 can compare a current image with the stored images with artifacts and identify the stored image that is most like the current image. The difference in pixel values between image of a stored pair determines the pixel value differentials that AI image processor adds to or subtracts from the pixel values of a current image from imaging module 602 to thereby produce a processed image that is essentially free of the ring artifact and can supply this processed image for display on display 110. In this manner, AI image processor 402 can replace the function of low reflective material or covering at surface 104D. Or, both ways of eliminating or reducing such ring artifacts can be used in the same endoscopic system 100.

It has also been unexpectedly discovered that another source of artifacts can be light that undergoes multiple reflections, that is, light from an LED in imaging module 602 that is reflected back (proximally) from tissue or liquid, then reflected distally from surface 104E and then reflected back proximally from tissue or liquid in the field of view of imaging module 602. Artifacts due to such multiple reflections may be more subtle that those due to reflections from surface 104D, but eliminating or reducing the multiple reflections can still help reduce interference with AEC, AGC, or ALS functions of imaging module 602 and result in clearer images that have greater diagnostic value.

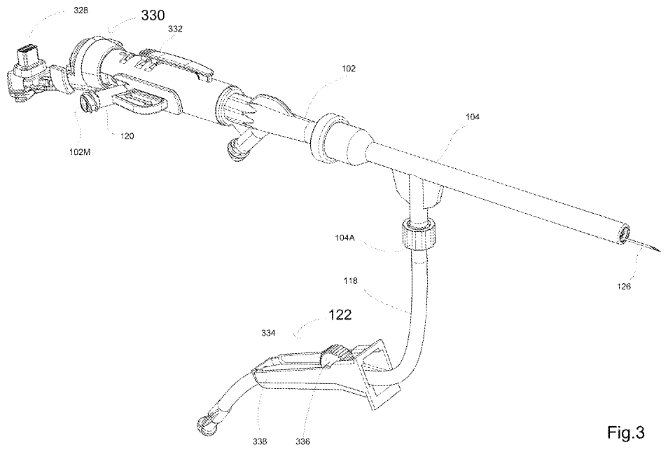

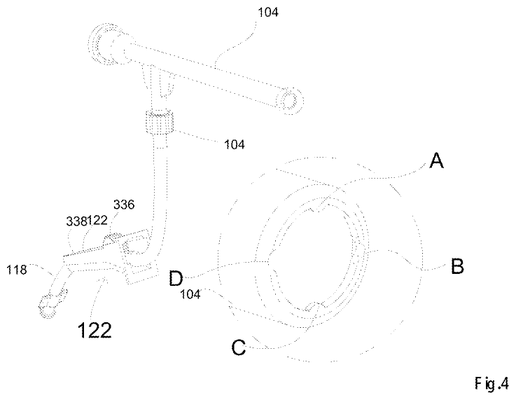

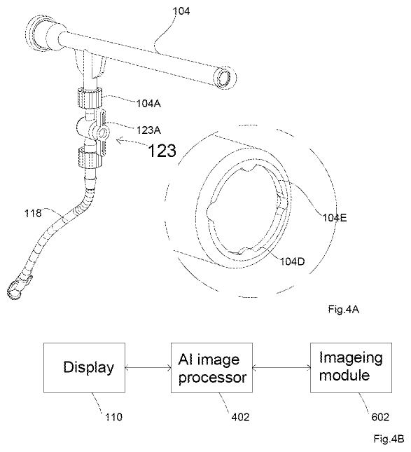

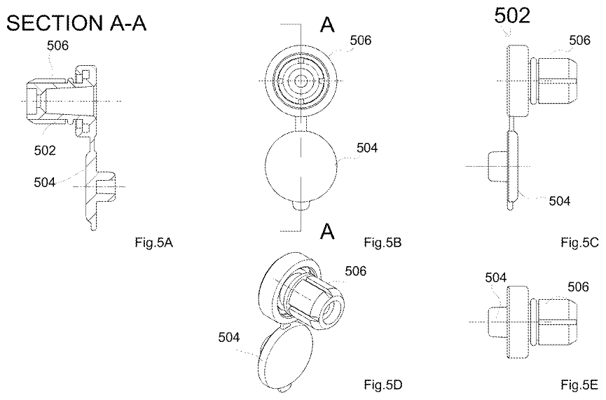

FIGS. 5A-5E illustrate a cap 502 configured to selectively close the proximal end of access sheath 104 when hinged lid 504 closes its proximal end (at right in FIG. 5A and left in FIG. 5C) of tubular cap body 506. Before a medical procedure the distal end (left in FIG. 5A and right in FIG. 5C) of cap body 506 is inserted in the proximal end of access sheath 104, with hinged lid 504 pivoted up to snap on and close off the proximal end of cap body 506. This protects the interior of access sheath 104 from foreign substances. In case a user inserts sheath 104 in a patient before inserting cannula 102 in sheath 104, cap 502 keeps fluid from flowing out the proximal end of sheath 104. To prepare for the medical procedure, the user unsnaps lid 504 from cap body 506 and pivots it down to thereby open the proximal end of cap body 506 for insertion of cannula 102 therethrough and into access sheath 104 so that sheath 104 can be inserted in the patient with live vision. FIG. 5A is a section at A-A in FIG. 5B, which is a view of the distal end of cap 502 with lid 504 open; FIG. 5C is a side view of cap 502 with lid 504 open; FIG. 5D is a perspective view; and FIG. 5E is a side view with lid 504 nearly but not completely closed.

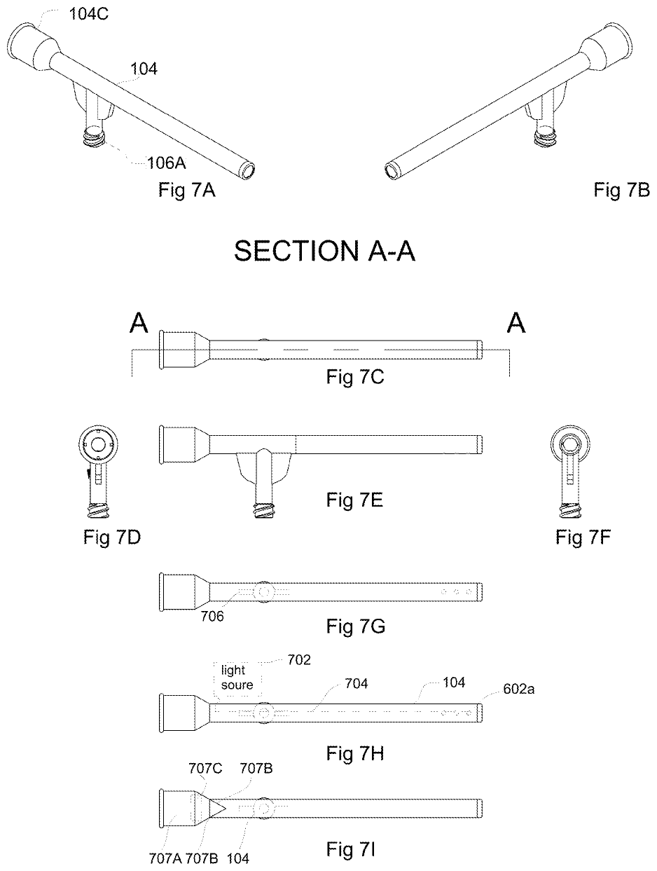

In some embodiments, instead of using a cap 502, sheath 104 can be provides with a duckbill valve 707 (FIG. 7I) that has a proximal end closed off with a membrane that is easily ruptured when a cannula 102 is introduced distally in sheath 104. Such membrane

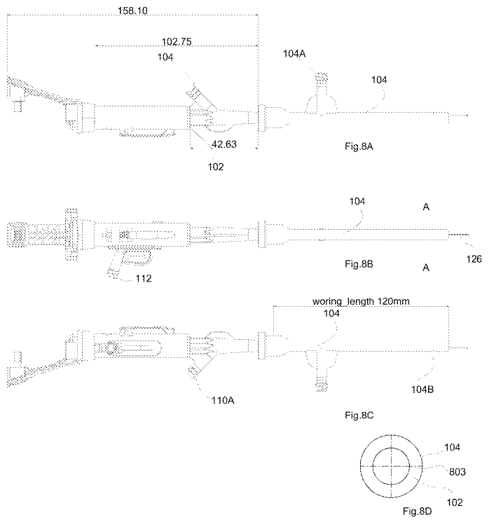

FIG. 6 is a perspective view of the distal portion of cannula 102 inside the distal portion of access sheath 104 in an access sheath, according to some embodiments. Sheath 104 has outflow ports 104B that are side facing in this example but can be distally facing in other examples. Cannula 102 has distally facing inflow ports 102B. Outflow here means flow in the proximal direction from ports 104B in access sheath, away from the patient and inflow means flow in the distal direction from cannula ports 102B into the patient. Fluid flow from ports 104B to port 104A is through space 803 (FIG. 8D) between cannula 102 and access sheath 104. Fluid flows from port 102A though one or more internal channels 102C in cannula 102 and out of ports 102B. The fluid flowing out of ports 102B flushes the field of view of imaging module 602 and is sucked away through ports 104B. Only one of the channels 102C is visible in FIG. 6, the other channel connects the other port 102B to port 102A. At selected times, such as during insertion of access sheath 104 into the patient, the user may elect to shut down outflow through ports 104B by closing off tube 118 with flow regulator 122. FIG. 6 illustrates injection needle 126 in its extended position; in its retracted position the distal end of needle 126 does not extend distally from access sheath 104.

Ports 102A and 102B can be used for the passing surgical devices to the target region instead of or in addition to providing fluid flow.

FIGS. 7A-7H show several views of access sheath 104, according to some embodiments. FIGS. 7A and 7B show two different perspective views and the funnel-shaped proximal end 104C of access sheath 104. FIG. 7C is a top view, FIG. 7D is a view in the distal direction, FIG. 7E is a left-side view, FIG. 7F is a view in the proximal direction, and FIG. 7G is a bottom view. FIG. 7H is a bottom view like FIG. 7C but additionally illustrates schematically an alternative way of providing light to an imaging module 602a that can be like module 602 but can be supplied with light from a light source 702 via one or more fiber optic strands 70. This light can be instead of or in addition to light from LEDs as in imaging module 602 of FIG. 6.

FIG. 7G shows a backflow restrictor 706 that is this example is an internal O-ring 706 though which cannula 102 passes when inserted in access sheath 104. O-ring is made of a resilient substance such as rubber and is configured to resist flow in the proximal direction from space 803 (FIG. 8D) between cannula 102 and axial sheath 104 and leak out through port 104A.

FIG. 7I is a sectional view of a proximal portion of sheath 104 taken along an axially extending central plane of sheath 104, and illustrates a backflow restrictor 707 that in this example is a duckbill valve with a larger diameter proximal portion 707A that keeps it from moving further distally in sheath 104 and a tapered distal portion 707B that is normally closed to prevent backflow from the proximal end of sheath 104, for example if a user inserts sheath 104 in patient without having first inserted cannula 102 in the sheath. Duckbill valve 707 is made of a flexible material such as rubber and has an open proximal end through which cannula 102 can be inserted distally in sheath 104. In distal motion into sheath 104, cannula spreads the distal portion 707B of duckbill 707 to pass through the duckbill valve but distal portion 707B remains biased radially inwardly around cannula 102 and keeps fluid from backflow in a proximal direction from space 803 (FIG. 8D) between cannula 102 and sheath 104. Preferably, endoscopic system 100 uses duckbill valve 707 instead of O-ring 706 but the O-ring may be used in some examples. As noted in connection with FIGS. 5A-5E, in some examples of endoscopic system 100 duckbill valve 707 may be provided with a membrane 707C that is easily ruptured by inserting cannula 102 in sheath 104. In such example, valve 707 with membrane 707C can keep the proximal end of sheath 104 from contamination in storing and handling.

FIGS. 8A-8C are a bottom view, left-side view, and a top view, respectively, of cannula 102 inserted in access sheath 104, and show an example of dimensions. FIG. 8D is a section at A-A of FIG. 8B and shows space 803 between cannula 102 and access sheath 104 through which fluid can flow between ports 104A and 104B. Space 803 extends axially from and includes the opening of port 104A into access sheath 104 and the distal end of cannula 102 when fully inserted into access sheath 104.

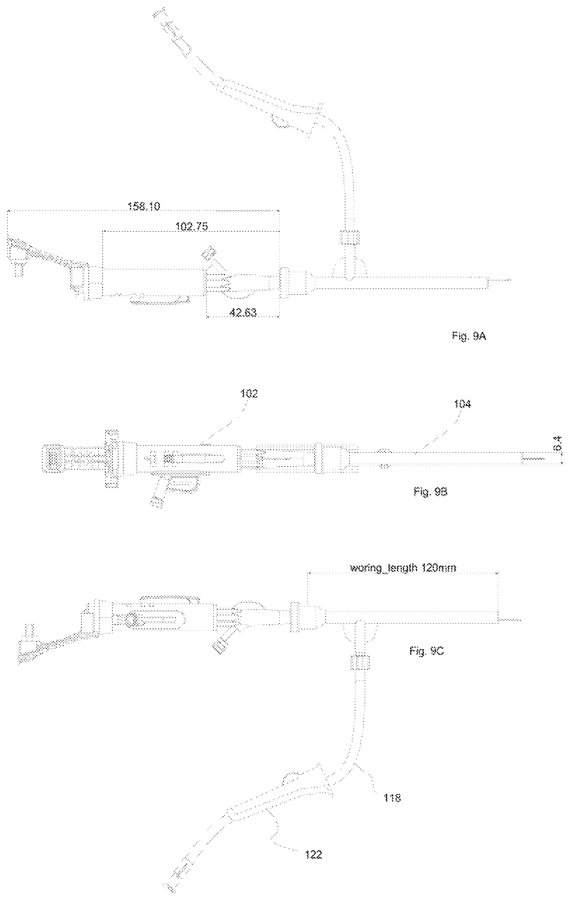

FIGS. 9A, 9B, and 9C are otherwise like FIGS. 8A-8C but show a different example of dimensions and show in addition tube 118 and flow regulator 122.

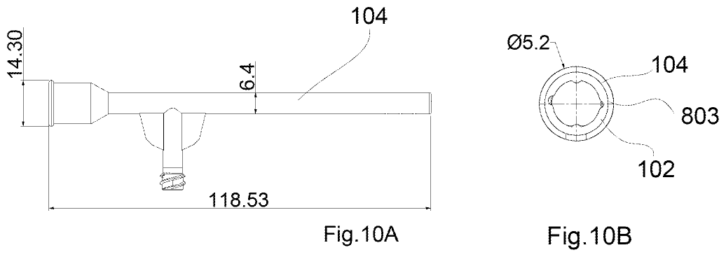

FIG. 10A is a left-side view of access sheath 104 and FIG. 10B is a cross-section at the distal end of access sheath 104, showing a different example of dimensions.

The dimensions shown in FIGS. 8A-C, 9A-C, and 10A-B are suitable examples, but other dimensions are contemplated for possible different medical needs or preferences.

An example of injecting medication in a patient's cavity under endoscopic guidance according to the disclosure above comprises: introducing access sheath 104, with cannula 102 inserted in sheath 104, into the patient's cavity such as the urethra or bladder; observing tissue distal of a distal end of the access sheath on display 114 mounted on handle 108 secured to the proximal end of cannula 102, wherein an imaging module at a distal end of the cannula inserted in the access sheath provides image data to said display; selectively flushing the cavity with a fluid entering the cannula through a port at a proximal portion thereof and exiting the cannula at one or more ports at a distal portion thereof with flushing fluid passing through one or more internal channels in the cannula; selectively aspirating the cavity through ports at a distal portion of the access sheath, a space between the cannula and the access sheath, and a port at a proximal portion of the access sheath; injecting medication at a first target site in the cavity with an injection needle that is permanently build inside the cannula, moves axially between extended and retracted positions, and has a distal end offset radially from a central axis of the cannula; and rotating the cannula relative to the sheath to thereby point a distal end of the injection needle at a second injection site and injecting medication at the second injection site. The access sheath that has a distal face made of or covered with a material that has low light reflection to thereby prevent or at least significantly reduce artifacts in images from said imaging module due to light reflected in the distal direction by said distal face of the sheath.

The benefits of the disclosed endoscopic system include: (1) access sheath 104 provides orientation landmarks ABCD within a target cavity that help a user visualize on screen 110 the position and orientation of the injection needle 126 and other elements of cannula 102 relative to the patient tissue of interest, (2) the distal face of access sheath 104 keeps cannula 102 from protruding distally from sheath 104 and thus keeps imaging module 602 or 602A from touching target tissue and thus ensures visualizing the tissue, (3) ports 102A and 102B and the internal channels connecting then, and ports 104A and 104B and space 803 provide inflow and/or outflow of fluid to and/or from the target region to flush or otherwise treat a target region in the patient, (4) one or both of the channels 102C connecting ports 102A and 102B can be used to pass surgical devices, and (5) access sheath 104 can provide illumination in addition to or instead of that provided by light sources in imaging module 126, for example by providing sheath 104 with light source 702 and fiber optic strands 704 to deliver light from an external light source to target tissue.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

1. A portable endoscopic system with a single-use cannula and a single-use access sheath, comprising:
a single-use, tubular access sheath 104 that has open proximal and distal ends;
a single-use cannula 102 that has a distal portion releasably sliding axially in said sheath to a working position in the sheath in which a distal end of the cannula is at a distal end of but inside the sheath and a proximal portion of the cannula extends proximally from the sheath;
wherein the outer diameter of the cannula portion that is in the sheath is sufficiently smaller than the inner diameter of the surrounding sheath to leave space 803 for axial fluid flow;
wherein said sheath has:
sheath ports 104A and 104B at its proximal and distal portions, respectively;
a back-flow restrictor 706 at its proximal end configured to resist proximal fluid flow from said space between the cannula and the sheath;
physical markings ABCD at its distal end arranged at selected angularly spaced positions; and
a funnel-shaped entrance into its open proximal end;
wherein said cannula has:
an imaging module 602, 602A at its distal end configured to produce image data for a field of view that includes said markings and further includes an imaged region distal from the sheath;
a permanently built-in, hollow injection needle 126 inside the cannula that has a distal end configured to move between an extended position in which it extend distally from the sheath and a retracted position in which it does not;
cannula ports 102A and 102B at proximal and distal portions of the cannula, respectively, and one or more internal channels coupling the cannula ports for fluid flow;
a syringe port 102M at the proximal portion of the cannula, coupled for fluid flow to a proximal end of the injection needle;
a syringe body 120 coupled with the injection needle and configured to move the needle axially between said retracted and extended positions thereof; and
a needle position lock 332 configured to selectively lock the injection needle against axial motion.

2. The endoscopic system of claim 1, in which the open distal end of the sheath has a radially inwardly facing surface 104D that is made of or is covered with a material characterized by low light reflectivity to thereby prevent or at least significantly reduce artifacts in images from said imaging module due to light reflected by said radially inwardly facing surface of the sheath.

3. The endoscopic system of claim 1, including an AI image processor configured to receive image data from said imaging module, identify a ring artifact therein due to reflection of light from a radially inwardly facing surface 104D at the distal end of the sheath, process the image data to remove or reduce the ring artifact, and supply processed image data to the display that is essentially free of said ring artifact.

4. The endoscopic system of claim 1, in which the open distal end of the sheath has distally facing surface 104E that is made of or covered with a material characterized by low light reflectivity to thereby prevent or at least significantly reduce artifacts in images from said imaging module due to light reflected by said distally facing distal face of the sheath.

5. The endoscopic system of claim 1, further including an aspiration source 124 and an aspiration tube 507 operatively coupling the aspiration source and said sheath port 104A at said proximal portion of the sheath, and a flow regulator 122 operatively coupled with said aspiration tube to selectively close said aspiration tube partly or completely.

6. The endoscopic system of claim 3, in which said flow regulator comprises a hand operated wheel 336 and an inclined raceway along which the wheel moves axially to thereby press the aspiration tube to selected degrees and restrict flow therethrough to a selected degree.

7. The endoscopic system of claim 3, in which said flow regulator comprises a hand operated stopcock.

8. The endoscopic system of claim 1, in which said sheath further including a tubular cap body 506 at said open proximal end of the sheath and a lid 504 hinged to the cap body to selectively close the cap body or open the cap body for passage therethrough of said cannula.

9. The endoscopic system of claim 1, further comprising a reusable portion to which said cannula mounts releasably and which comprises a display operatively coupled with said imaging module to display images taken with the imaging module, and a handle on which the display is mounted.

10. The endoscopic system of claim 1, in which a distal end of said injection needle is offset from a central axis of said cannula and said cannula is configured to rotate in said access sheath which the sheath remains stationary relative to space of to a patient, wherein the needle points to different injection sites for different angular positions of the cannula relative to the sheath.

11. The endoscopic system of claim 1, in which the portion of the access sheath between the funnel-shaped proximal end and the distal end thereof is approximately 10 cm.

12. The endoscopic system of claim 1, in which the cannula portion that is in the access sheath is no longer than 11.5 cm.

13. A portable endoscopic system with a single-use cannula and a single-use access sheath, comprising:
a single-use, tubular access sheath 104 that has open proximal and distal ends;
a single-use cannula 102 that has a distal portion releasably sliding axially in said sheath to a working position in which a distal end of the cannula is at a distal end of but inside the sheath and a proximal portion of the cannula extends proximally from the sheath;
wherein the outer diameter of the cannula portion that is in the sheath is sufficiently smaller than the inner diameter of the surrounding sheath to leave space 803 for axial fluid flow;
wherein said sheath has:
sheath ports 104A and 104B at its proximal and distal portions, respectively; and
physical markings at its distal end arranged at selected angularly spaced positions;
wherein said cannula has:
an imaging module 602, 602A at its distal end configured to produce image data for a field of view that includes said markings and further includes a target region distal from the sheath;
a hollow injection needle 126 inside the cannula that has a distal end configured to move between an extended position in which it extend distally from the sheath and a retracted position;
cannula ports 102A and 102B at proximal and distal portions of the cannula, respectively, and one or more internal channels coupling the cannula ports for fluid flow;
a syringe port 102M at the proximal portion of the cannula, coupled for fluid flow to a proximal end of the injection needle; and
a needle position lock 332 configured to selectively lock the injection needle against axial motion.

14. The endoscopic system of claim 13, in which the open distal end of the sheath has a radially inwardly facing surface 104D that is made of or is covered with a material characterized by low light reflectivity to thereby prevent or at least significantly reduce artifacts in images from said imaging module due to light reflected by said radially inwardly facing surface of the sheath.

15. The endoscopic system of claim 13, including an AI image processor configured to receive image data from said imaging module, identify a ring artifact therein due to reflection of light from a radially inwardly facing surface 104D at the distal end of the sheath, process the image data to remove or reduce the ring artifact, and supply processed image data to the display that is essentially free of said ring artifact.

16. The endoscopic system of claim 13, in which the open distal end of the sheath has a distally facing surface 104E that is made of or covered with a material characterized by low light reflectivity to thereby prevent or at least significantly reduce artifacts in images from said imaging module due to light reflected by said distally facing surface of the sheath.

17. The endoscopic system of claim 13, further including a syringe body that connects to a proximal end of said injection needle and is configured for manual operation to move said needle axially between said extended and retracted positions.

18. The endoscopic system of claim 13, in which said cannula is configured to rotate relative to the access sheath.

19. The endoscopic system of claim 13, in which the distal end of said injection needle is offset from a central axis of the cannula and the cannula is configured for rotation in the axial sheath, whereby different angular position of the cannula relative to the sheath cause the distal end of the needle to point at different injection sites.

20. A method of injecting medication in a patient's cavity under endoscopic guidance, comprising:
introducing an access sheath into the patient's cavity;
observing tissue distal of a distal end of the access sheath on a display mounted on a handle secured to a proximal end of a cannula, wherein an imaging module at a distal end of the cannula inserted in the access sheath provides image data to said display;
selectively flushing the cavity with a fluid entering the cannula through a port at a proximal portion thereof and exiting the cannula at one or more ports at a distal portion thereof with flushing fluid passing through one or more internal channels in the cannula;
selectively aspirating the cavity through ports at a distal portion of the access sheath, a space between the cannula and the access sheath, and a port at a proximal portion of the access sheath;
injecting medication at a first target site in the cavity with an injection needle that is permanently build inside the cannula, moves axially between extended and retracted positions, and has a distal end offset radially from a central axis of the cannula; and
rotating the cannula relative to the sheath to thereby point a distal end of the injection needle at a second injection site and injecting medication at the second injection site.

21. The method of claim 18, in which the introducing step comprises introducing an access sheath with open distal end that has a radially inwardly facing surface 104D made of or covered with a material characterized by low light reflectivity to thereby prevent or at least significantly reduce artifacts in images from said imaging module due to light reflected by said radially inwardly facing surface of the sheath.

22. The method of claim 20, in which the introducing step comprises introducing an access sheath with an open distal end that has a distally facing surface 104E made of or covered with a material characterized by low light reflectivity to thereby prevent or at least significantly reduce artifacts in images from said imaging module due to light reflected by said distally facing surface of the sheath.

23. The method of claim 20, including AI processing of image data from said imaging module to identify a ring artifact therein due to reflection of light from a radially inwardly facing surface 104D at the distal end of the sheath and supplying for display processed image data from which said ring artifact has been essentially removed.

24. The method of claim 18, including observing angular position of the cannula relative to the sheath on the display indicated by images of markings angularly spaced around a distal end of the sheath that is within the field of view of the imaging module.

25. The method of claim 18, in which the sheath remains at the same angular position relative to the cavity while the cannula rotates between the first and second injections.

26. The method of claim 18, in which the sheath remains with said port at the proximal portion of the sheath pointing down during the first and the second injections.

27. The method of claim 18, in which the sheath is inserted in a patient's urethra to the patient's bladder.
